(19) United States
(12) Patent Application Publication
(10) Pub. No.: US 2009/0024028 A1
(43) Pub. Date: Jan. 22, 2009

(54) METHOD AND SYSTEM FOR EVALUATING IMAGES

(75) Inventors: Michael Joseph Washburn, Brookfield, WI (US); Markus Wilhelm Marquart, Eching (DE)

Correspondence Address: PETER VOGEL;GE HEALTHCARE, 20225 WATER TOWER BLVD., MAIL STOP W492, BROOKFIELD, WI, 53045, US

(73) Assignee: General Electric Company, Schenectady, NY (US)

(21) Appl. No.: 11/779,610

(22) Filed: Jul. 18, 2007

(51) Int. Cl. A61B 8/00

(52) U.S. Cl. 600/437

(57) ABSTRACT

A method and system for evaluating images is disclosed herein. A method for evaluating non-ultrasound images using an ultra-sound imaging system comprises: obtaining plurality of ultrasound images using the ultrasound imaging system and fetching a plurality of non-ultrasound DICOM images to the ultrasound imaging system. Once the images are obtained, ultrasound measurements are performed in the ultrasound and non-ultrasound images using the ultrasound imaging system to obtain at least one predefined parameter from the ultrasound and non-ultrasound images. Once the predefined parameters are obtained, they are compared to interpret the results.

### FIELD OF THE INVENTION

This invention relates generally to image evaluation methods and systems, and more particularly to, a method and system for evaluating non-ultrasound images using an ultrasound imaging system.

### BACKGROUND OF THE INVENTION

In medical imaging applications, images of an anatomy are often obtained and stored in different medias for re-using the images at a later stage. However sometimes, body parts might change in their structure or shape from a prior point in time. In many instances it is desired to identify these changes in the bodyparts. Taking images at certain intervals and comparing the images to identify the changes in the body parts achieve this. Many times the images are taken using different imaging systems and are measured and processed in different systems. Thus the evaluation of the images is often done by different people, using different systems.

Considering an example a tumor growth, the volume of a tumor may change in response to a treatment. To determine the efficacy of the treatment, the physician needs to identify whether the size or volume of the tumor has been affected by the treatment. This is achieved by taking images of the tumor during the course of treatment at some desired intervals. However due to various reasons it may not be feasible to take the images of the patient every time using a magnetic resonance (MR) or computed tomography (CT) imaging system. Generally, pre-acquired images of an anatomy or tumor are taken before the treatment is initiated using any imaging system such as CT, MR or positron emission tomography (PET). However during follow up of the treatment, the images could be taken using an ultrasound imaging system as the radiation exposure to the patient and the cost of scanning will be less compared to these other imaging techniques. The pre-acquired images may be stored in DICOM format in the imaging system itself or in any other image storing devices. During the follow up, the physician evaluates the pre-acquired images, which are stored in any imaging system or in any other storage device, and the latest images of the anatomy available in the ultrasound imaging system. Even though all the images are in DICOM format, they are stored in different imaging systems and could be stored in different formats. For example, the pre-acquired images of the tumor may be stored in a picture archiving and communication system (PACS). For determining the changes in the size or shape of the tumor, the image of the tumor needs to be measured. For measuring the images in the PACS system, the calculation package available in the PACS system or any other similar package compatible with the PACS system is used, and for ultrasound images in the ultrasound imaging system, the calculation package available in the ultrasound imaging system is used. The calculation packages available in different systems may be different and based on that there could be differences in the measurements. These measured results obtained from different imaging systems are later compared to identify the changes in the size or volume of the tumor.

Further since the measurements are done using two different systems, the comparison is not done in the same system and may result in some inaccuracies. Most of the time different people do the measurements as the images are stored in different systems and hence the judgment of the clinician who performs the measurements may affect the final result.

In some instances the results of the measurement may not be available in the imaging system as the measurements are performed in different systems than in the imaging system. So if the physician needs to analyze the result and take some action, it will be difficult to do so as the results may not be readily available in the ultrasound scanning system.

Thus it will be beneficial if a single person using a single system is enabled to evaluate the images obtained by different imaging systems. Thus there exists a need to provide a method and system for evaluating non-ultrasound images using an ultrasound imaging system.

### SUMMARY OF THE INVENTION

The above-mentioned shortcomings, disadvantages and problems are addressed herein which will be understood by reading and understanding the following specification.

The present invention provides a method of evaluating non-ultrasound images using an ultrasound imager. The method comprises the steps of: obtaining a plurality of ultrasound images using the ultrasound imager, and fetching a plurality of DICOM images including non-ultrasound images to the ultrasound imager. The method further includes performing ultrasound measurements on the ultrasound images and the non-ultrasound images using the ultrasound imager, obtaining at least one predefined parameter from each of the ultrasound images and the non-ultrasound images, and comparing the predefined parameter obtained from the ultrasound images with the predefined parameter obtained from the non-ultrasound images.

In another embodiment, an ultrasound imaging system is provided. The ultrasound imaging system comprises: an ultrasound imager generating ultrasound images and an interface unit configured to receive pre-acquired DICOM images including non-ultrasound images. The ultrasound imaging system further comprises a measuring unit configured to perform ultrasound measurements on the ultrasound images and the DICOM images and a comparison unit to evaluate the results of the measuring unit. In an embodiment the measuring unit is further configured to obtain at least one predefined parameter from the ultrasound images and the pre-acquired DICOM images, and the predefined parameter is selected from a group of parameters including size, shape, distance, volume, circumference, area, distance ratio, area ratio, angle and percent stenosis of a body part.

In yet another embodiment, a multi-modality ultrasound system is disclosed. The multi-modality ultrasound system comprises: an imaging system providing a plurality of non-ultrasound images and an ultrasound imaging system providing ultrasound images. The ultrasound imaging system is configured to receive the non-ultrasound images and perform ultrasound measurements on the non-ultrasound images.

Various other features, objects, and advantages of the invention will be made apparent to those skilled in the art from the accompanying drawings and detailed description thereof.

### BRIEF DESCRIPTION OF THE DRAWINGS

FIG. 1 is a flowchart illustrating the method of evaluating non-ultrasound images using an ultrasound imaging system;

FIG. 2 is a block diagram of an ultrasound imaging system as described in an embodiment of the invention; and

FIG. 3 is a block diagram of a multi-modality ultrasound system as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments a method for evaluating images acquired by different imaging systems is disclosed. The images of an anatomy acquired by different imaging system are brought together to a single system and then evaluated.

In various embodiments, a method and system for evaluating non-ultrasound images using an ultrasound imaging system is provided. The method incorporates configuring an ultrasound imaging system to receive any DICOM images including non-ultrasound images and performs ultrasound measurements and calculations on the non-ultrasound images.

In an embodiment the ultrasound imager performs ultrasound measurements and calculations on ultrasound and non-ultrasound images to obtain at least one predefined parameter of the imaged anatomy. The ultrasound imager is configured to receive non-ultrasound images.

In an embodiment a method of measuring changes in a body structure or shape using an ultrasound imaging system is disclosed. The ultrasound imaging system computes the difference in the imaged anatomy using pre-acquired non-ultrasound images and the latest ultrasound images. In an example, the method may be used to identify the changes in shape, form, position and configuration of a lesion or tumor.

In an embodiment the invention provides an integrated workstation to process non-ultrasound images. The integrated workstation will have an ultrasound imaging system and any other imaging system. The other imaging system may be a PET, CT, X-ray, or MR imaging system, or an imaging system of any other imaging modality.

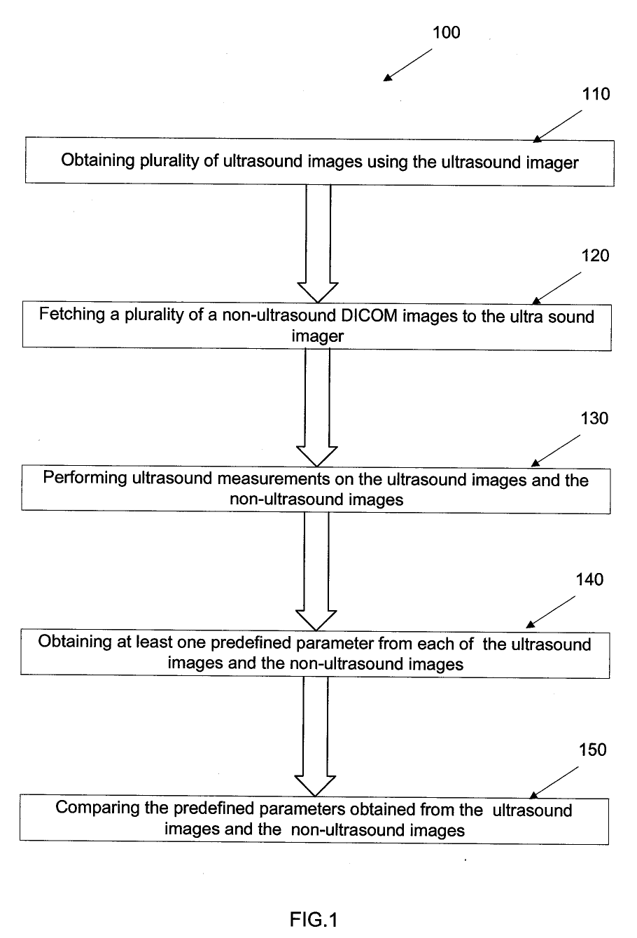

FIG. 1 is a flowchart illustrating a method of evaluating non-ultrasound images using an ultrasound imaging system in accordance with one embodiment of the invention. The method 100 is explained in reference to medical imaging. At step 110, the ultrasound imaging system obtains a plurality of ultrasound images of a patient using an ultrasound imager. The ultrasound images obtained can be stored in the ultrasound imaging system or in any other image storing devices. At step 120, a plurality of DICOM images including non-ultrasound images are fetched into the ultrasound imager. The ultrasound imaging system needs to be configured to receive any DICOM images including non-ultrasound images. In an example, these DICOM images are pre-acquired non-ultrasound images acquired by any non-ultrasound imaging system such as CT, PET, X-ray, MR, etc. system. Also, the pre-acquired non-ultrasound images can be fetched from a reviewing station, PACS or any other image-storage or archival device. At step 130, the ultrasound imaging system performs ultrasound measurements and calculation. The ultrasound measurements and calculations are performed on the ultrasound images and the non-ultrasound images. The ultrasound measurements and calculations are performed to obtain predefined parameters such as size, shape, distance, volume, circumference, area, distance ratio, area ratio, angle and percent stenosis etc of imaged anatomy. At step 140, at least one predefined parameter is obtained from the non-ultrasound images and the ultrasound images. The predefined parameters include size, shape, volume, circumference, area, distance ratio, area ratio, angle and percent stenosis, etc., of a body part. However the parameters need not be limited to these. The predefined parameters are identified from the ultrasound images and non-ultrasound images by the ultrasound imaging system. At step 150, the predefined parameters obtained from the non-ultrasound images and the ultrasound images are compared. By comparing the predefined parameters, the result is interpreted to identify a change in the body part. The compared results can be displayed on a display device along with non-ultrasound images and the ultrasound images together in a side-by-side display.

In an embodiment the invention discloses a method of identifying changes in a bodypart. The method includes pre-acquiring a plurality of images of the body part using a non-ultrasound imaging system and acquiring a plurality of latest images of the body part using an ultrasound imaging system. The pre-acquired non-ultrasound images are fed to the ultra-sound imaging system and ultrasound measurements and calculations are performed for obtaining a predetermined parameter of the bodypart. The ultrasound measurements and calculations are performed on the ultrasound as well as non-ultrasound images using the calculation package available on the ultrasound imaging system. The predetermined parameter of the body part obtained from the ultrasound images and non-ultrasound images are compared and form the comparison result from which the change in the body part is interpreted.

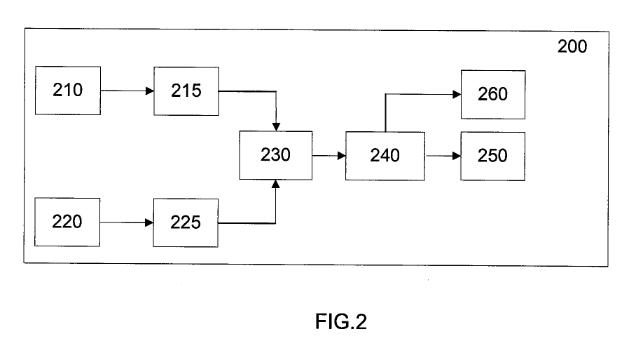

FIG. 2 is a block diagram of an ultrasound imaging system as described in an embodiment of the invention. The ultrasound imaging system 200 is configured to evaluate non-ultrasound images. The system 200 includes an ultrasound imager 210 configured to generate ultrasound images. The ultrasound imager 210 generates the ultrasound images 215 of the desired body part and the ultrasound images may be stored in the imaging system. The ultrasound imaging system 200 is provided with an interface 220. The interface 220 configures the ultrasound imaging system 200 to receive any DICOM images including non-ultrasound images 225. The interface 220 receives any DICOM images including non-ultrasound images from any non-ultrasound imaging systems such as X-ray, CT, PET, MR etc or from any review workstations, PACS system or any other image storage devices. In an embodiment the DICOM images could be pre-acquired non-ultrasound images of an anatomy. The ultrasound images 215 from the ultrasound imager 210 and the non-ultrasound images 225 received from other imaging systems through the interface 220 are fed to a measuring unit 230. The measuring unit 230 is configured to perform ultrasound measurements and calculations on the ultrasound and non-ultrasound images. The measurement unit 230 includes a set of instructions configured to obtain at least one predefined parameter from the ultrasound images and the non-ultrasound images. The predefined parameter includes size, shape, distance, volume, circumference, area, distance ratio, area ratio, angle and percent stenosis etc of imaged anatomy. At least one predefined parameter is identified from the ultrasound images and the non-ultrasound images using the measuring unit 230. The identified parameters from the ultrasound and non-ultrasound images are compared in a comparison unit 240. This facilitates measuring the ultrasound and non-ultrasound images using the same system, comparing the results of measurement in the same system, and displaying the images and the result in the same system. Once compared, the result of comparison unit 240 can interpret the change in the imaged anatomy. The images including the comparison results can be displayed on a display 250 and/or can be stored in a memory 260. The images and/or measurements thus stored in the memory 260 can be used for different diagnostic purposes including determining the changes in the structure or shape of a bodypart. The images may be stored in the memory and may be later displayed by any displaying devices in different formats.

Considering the example of imaging a tumor in a body, initially the images of the anatomy is taken using an imaging system such as MR, CT, PET, etc. These images may be stored in the imaging system itself or can be stored in a PACS. After a desired interval the images of the tumor need to be taken again. The images of the tumor are taken using an ultrasound imaging system. Once the latest images of the tumor are taken, the pre-acquired images are fetched into the ultrasound imaging system. The ultrasound imaging system is configured to receive any DICOM images. The pre-acquired non-ultrasound images and the ultrasound images are available in the ultrasound imaging system and ultrasound measurements and calculations are performed on these images. The ultrasound measurements and calculations are done to measure various features of an imaged anatomy having tumor. The features include shape, form, position and configuration of a lesion or tumor. These features are defined by determining a predefined parameter from the ultrasound and non-ultrasound images. The parameter may include size, shape, distance, volume, circumference, area, distance ratio, area ratio, angle and percent stenosis, etc. of imaged anatomy. Once the parameters from the ultrasound and non-ultrasound images are identified, the imaging system compares the results and the change in the tumor is identified. The results can then be displayed to the user.

In an example the distance between two pixels in an ultrasound image and non-ultrasound image can be measured and compared using an ultrasound imaging system.

In an example a volume from one or more distance is computed for both ultrasound and non-ultrasound images using an ultrasound imaging system.

However the applications of the evaluation method and system need not be limited to medical imaging applications and to the examples mentioned above.

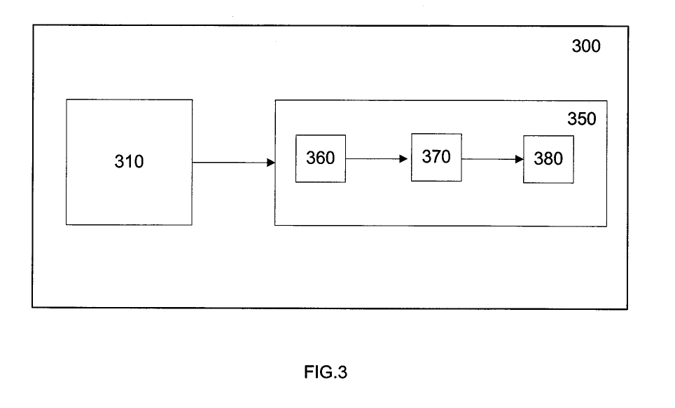

FIG. 3 is a block diagram of a multi-modality ultrasound system as described in an embodiment of the invention. The multi-modality ultrasound system 300 is an integrated ultrasound imaging system integrating different imaging modalities for evaluating the images. The multi-modality ultrasound system 300 includes an imaging system 310 generating DICOM images. In an embodiment the imaging system 310, includes a non-ultrasound imaging system such as MR, CT, PET, X-Ray, etc. generating non-ultrasound images in DICOM format. The multi-modality ultrasound system 300 includes an ultrasound imaging system 350, which is configured to process or evaluate the images. The ultrasound imaging system includes an imager 360, which generates the ultrasound images of an anatomy. Generally the ultrasound images are acquired to give the latest image of the anatomy. The ultrasound images may be stored in the ultrasound imaging system 350. The ultrasound imaging system 350 further includes a processor 370, which configures the ultrasound imaging system 350 to receive any DICOM images including non-ultrasound images from the imaging system 310. The ultrasound imaging system 350 has the ultrasound images as well as the non-ultrasound images. The processor 370 performs ultrasound measurements and calculations on the ultrasound and non-ultrasound images. The ultrasound measurements and calculations are performed to evaluate the images. The evaluation includes obtaining at least one predefined parameter from the ultrasound and non-ultrasound images. The parameter may include size, shape, distance, volume, circumference, area, distance ratio, area ratio, angle and percent stenosis, etc. of imaged anatomy. Once the desired parameters are obtained from the ultrasound and non-ultrasound images, the obtained parameters are compared. Based on the comparison results, the changes in the images of an anatomy can be identified. The ultrasound imaging system 350 can have a display or monitor 380 for displaying the images in different formats.

In an embodiment, the ultrasound images acquired by a first ultrasound imaging system could be fetched to a second ultrasound imaging system and a single operator could evaluate the images using the same system.

In an embodiment if the measurements are done side-by-side then the results can be stored for documentation purpose as well.

Some of the advantages of the invention include the ability to remove or improve potential variations in the evaluation results obtained from a pre-acquired volume acquired by a non-ultrasound imaging system and an ultrasound image acquired by an ultrasound imaging system. The variations that are removed or improved include variations in operator decisions. If the ultrasound images and non-ultrasound images are measured in different imaging system, the operator who performs the measurement and calculation on the images could be different and this may result in some discrepancies. For example, while identifying the edge of a tumor, the judgment of each operator who identifies the edges will play an important role and this may affect the final result. Since according to one aspect of the method the non-ultrasound images are fetched to the ultrasound imaging system, the evaluation is performed on the non-ultrasound images and the ultrasound images within the ultrasound imaging system. Another variation that is removed or improved is variation in the calculation formulas or methods. Since the calculation and measurements are performed by the same system, the package used for performing these measurements remains the same and hence variations due to difference in calculation methods or packages are eliminated.

Another advantage of various embodiments of the invention includes the ability to store the resulting measurements in a single image. Since the results of the comparison of the images are available in the same system along with the ultrasound and non-ultrasound images, the images can be displayed along with the results.

Another advantage of the approach is that, by showing images side by side, it helps to assure that the same view of the same anatomy is being measured. In some embodiments, this is being done is real time during the ultrasound acquisition so adjustments can immediately be made if needed.

The ultrasound measurements of the invention need not be limited to the examples mentioned in the specification. Also the invention is applicable to different ultrasound applications such as volume-guided ultrasound, echocardiography, Gynecology, Endocrinology etc to mention a few.

Thus various embodiments of the invention describe method and system for evaluating images acquired using different imaging systems. In an embodiment the invention discloses a method and system to evaluate non-ultrasound images using an ultrasound imaging system.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method of evaluating non-ultrasound images using an ultrasound imager comprising the steps of:
obtaining a plurality of ultra sound images using the ultrasound imager;
fetching a plurality of DICOM images including non-ultrasound images to the ultrasound imager;
performing ultrasound measurements on the ultrasound images and the non-ultrasound images using the ultrasound imager;
obtaining at least one predefined parameter from each of the ultrasound images and the non-ultrasound images; and
comparing the predefined parameter obtained from the ultrasound images with the parameter obtained from the non-ultrasound images.

2. A method as in claim 1, wherein the step of fetching comprises: fetching a plurality of pre-acquired DICOM images including non-ultrasound images.

3. A method as in claim 1, wherein the step of fetching comprises: configuring the ultrasound imager to receive DICOM images including non-ultrasound images.

4. A method as in claim 1, wherein the step of obtaining at least one predefined parameter comprises: obtaining the predefined parameter selected from a group of parameters including size, shape, distance, volume, circumference, area, distance ratio, area ratio, angle and percent stenosis of an imaged anatomy.

5. A method as in claim 1, wherein the ultrasound measurements are performed during acquisition of the ultrasound images.

6. A method as in claim 1, wherein the step of comparing comprises: displaying the ultrasound images and the non-ultrasound images side by side.

7. A method as in claim 1, wherein the step of comparing comprises: interpreting a change in a body part from results of the comparison.

8. An ultrasound imaging system comprising:
an ultrasound imager generating ultra-sound images;
an interface unit configured to receive pre-acquired DICOM images including non-ultrasound images;
a measuring unit configured to perform ultrasound measurements on the ultrasound images and the DICOM images;
a comparison unit to evaluate the results of the measuring unit.

9. An ultrasound imaging system as in claim 8, wherein the interface unit includes a set of instructions loaded in the imaging system for configuring the ultrasound imager to receive DICOM images.

10. An ultrasound imaging system as in claim 9, wherein the interface unit is configured to accept images from a source of images including an imaging system, a picture archiving and communications system (PACS), a review workstation and a memory device.

11. An ultrasound imaging system as in claim 8, wherein the measuring unit includes a set of instructions loaded in the imaging system for performing ultrasound measurements and calculations on DICOM images.

12. An ultrasound imaging system as in claim 11, wherein the measuring unit is further configured to obtain at least one predefined parameter from the ultrasound images and the pre-acquired DICOM images.

13. An ultrasound imaging system as in claim 12, wherein the predefined parameter is selected from a group including size, shape, distance, volume, circumference, area, distance ratio, area ratio, angle and percent stenosis of a body part.

14. An ultrasound imaging system as in claim 13, wherein the comparison unit is configured to compare the predefined parameter obtained from the ultrasound images and the pre-acquired DICOM images.

15. An ultrasound imaging system as in claim 8, further comprising a display unit configured to display the ultrasound images and the pre-acquired DICOM images side by side along with comparison results.

16. A multi-modality ultrasound system comprising:
an imaging system providing a plurality of non-ultrasound images; and
an ultrasound imaging system providing ultrasound images;
wherein the ultrasound imaging system is configured to receive the non-ultrasound images and perform ultrasound measurements on the non-ultrasound images.

17. A system as in claim 16, wherein the imaging system is one of a magnetic resonance (MR) system, a computed tomography (CT) system, an X-Ray system, and a positron emission tomography (PET) system.

18. A system as in claim 16, wherein the ultrasound imaging system includes an ultrasound imager configured to generate ultrasound images.

19. A system as in claim 18, wherein the ultrasound imaging system further includes a processor configuring the ultrasound imager to receive the non-ultrasound images.

20. A system as in claim 19, wherein the processor is configured to perform ultrasound measurements on the ultrasound images and non-ultrasound images.

21. A system as in claim 20, wherein the processor is further configured to obtain at least one predefined parameter from the ultrasound images and the non-ultrasound images and to compare the obtained predefined parameters.

22. A system as in claim 16, wherein the ultrasound images and the non-ultrasound images are in DICOM format.
